(19) United States
(12) Patent Application Publication
(10) Pub. No.: US 2022/0257541 A1
(43) Pub. Date: Aug. 18, 2022

(54) Lacosamide for the Treatment of ADD/ADHD

(71) Applicant: Hector Mario Sanchez, Santo Domingo (DO)

(72) Inventor: Hector Mario Sanchez, Santo Domingo (DO)

(21) Appl. No.: 17/628,814

(22) PCT Filed: Jul. 31, 2020
(86) PCT No.: PCT/EP2020/071735

§ 371 Date: Jan. 20, 2022

(30) Foreign Application Priority Data
Aug. 2, 2019 (GB) 1911056.8

(51) Int. Cl. A61K 31/165; A61P 25/00

(52) U.S. Cl. CPC A61K 31/165; A61P 25/00

(57) ABSTRACT

The present invention relates to the treatment of attention deficit hyperactivity disorder (ADHD) or attention deficit disorder (ADD) using lacosamide or functionally equivalent analogues thereof. The treatment may result in one or more of: (i) an improvement of cognitive function; (ii) an improvement of cognitive performance; (iii) a reduction in hyperactivity; (iv) an improvement in the circadian rhythm and/or sleep quality; and/or (v) an improvement in mood.

### FIELD OF THE INVENTION

The present invention relates to the treatment of Attention Deficit Disorder (ADD) and/or Attention Deficit Hyperactivity Disorder (ADHD). Treatment uses “non-stimulant” compounds, such as lacosamide or functionally equivalent analogues thereof.

### BACKGROUND TO THE INVENTION

Attention Deficit Hyperactivity Disorder (ADHD) is a neurological disorder characterised by problems paying attention and difficulty controlling behaviour which is not appropriate for a person's age. Based on the Diagnostic and Statistical Manual (DSM) criteria, there are three sub-types of ADHD:
(i) ADHD predominantly inattentive type (ADHD-PI or ADHD-I) presents with symptoms including being easily distracted, forgetful, daydreaming, disorganization, poor concentration, and difficulty completing tasks;
(ii) ADHD, predominantly hyperactive-impulsive type (ADHD-PH or ADHD-HI) presents with excessive fidgetiness and restlessness, hyperactivity, difficulty waiting and remaining seated and immature behaviour; destructive behaviours may also be present; and
(iii) ADHD, combined type (ADHD-C) which is a combination of subtypes (i) and (ii) above.

ADHD predominantly inattentive type (ADHD-PI or ADHD-I) lacks a hyperactivity component and may also be known as Attention Deficit Disorder (ADD).

The underlying cause of ADD/ADHD is not well understood. It has been hypothesized that it is related to deficiencies in dopamine production/firing and/or noradrenaline production/firing.

As a consequence, it is conventional in the art to treat neurological disorders using molecular stimulants. In the case of ADD/ADHD, methylphenidate is predominantly used. Molecular stimulants (such as methylphenidate) act by stimulating/modulating neurotransmitter release or recapture transporters in the brain. In the specific case of methylphenidate, this compound inhibits norepinephrine and dopamine reuptake by blocking both the norepinephrine transporter and dopamine transporter in brain cell membranes. This prevents dopamine clearance from synapses, elevating dopamine levels in the synapse and increasing dopamine signalling in the brain. However, the US Food and Drug Administration has now imposed a warning on methylphenidate packaging warning that this drug can cause addiction and the French Agence Nationale de Sécurité du Medicament has published a detailed report on the broad array of secondary symptoms caused by the medium to long term use of methylphenidate including insomnia, seizures, tic disorders, anxiety, irritability, aggressiveness, depression and mood swings (“Méthylphénidate: données d'utilisation et de sécurité d'emploi en France”, Juillet 2013).

It has also been demonstrated that one third of the children who take methylphenidate develop symptoms of obsessive-compulsive behaviour (“Methylphenidate-induced obsessive-compulsive symptoms: A case report and review of literature”; Jhanda et al.; J Pediatr Neurosci. 2016; 11(4): 316-318).

In addition, this drug may cause hallucinations, suicidal thoughts and psychotic behaviour that may result in aggressive and violent episodes. According to the U.S. Food and Drug Administration, it often leads to gastrointestinal problems and loss of appetite, endocrinology disorders that cause weight loss or other growth issues, cardiac events such as tachycardia, ventricular fibrillation and in some cases an implication in sudden death (“Weight, Height, and Body Mass Index in Patients with Attention-Deficit/Hyperactivity Disorder Treated with Methylphenidate”, Díez-Suárez et al.; J Child Adolesc Psychopharmacol. 2017 Aug. 17).

### SUMMARY OF THE INVENTION

Stimulants such as methylphenidate may appear to quickly control one or more of the symptoms of ADD/ADHD, however they only treat the symptoms in the short-term without addressing the underlying etiology. Such stimulants do not therefore provide a suitable long-term treatment solution.

The present inventor has now surprisingly discovered that certain compounds which are active in the brain but which do not stimulate neurotransmitter release or recapture transporters in the brain can be used to treat ADD/ADHD. This “non-stimulant” approach provides a progressive and long-lasting stabilisation of the symptoms of ADD/ADHD. This is indicative that these compounds functionally address the etiological source of the disorder.

For example, this approach may result in one or more or all of the following:

(i) an improvement of cognitive function;

(ii) an improvement of cognitive performance;

(iii) a reduction in hyperactivity;

(iv) an improvement in the circadian rhythm and/or sleep quality; and/or

(v) an improvement in mood.

“Non-stimulant” compounds are known in the art to treat conditions such as epilepsy and bipolar disorder. An exemplary compound is lacosamide. Lacosamide's chemical name is (2R)-2-(acetylamino)-N-benzyl-3-methoxypropanamide and it has the following structure:

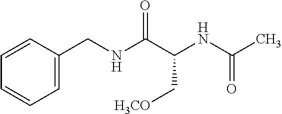 [SMILES: COC[C@@H](NC(C)=O)C(=O)NCc1ccccc1]

Lacosamide is a well-known anticonvulsant compound approved for the adjunctive treatment of partial-onset seizures and neuropathic pain.

Lacosamide is a functionalized amino acid that is believed to act through voltage-gated sodium channels (“Current understanding of the mechanism of action of the antiepileptic drug lacosamide” Rogawski et al., 2015, Epilepsy Research, 110: 189-205). Lacosamide enhances the slow inactivation of voltage-gated sodium channels without affecting the fast inactivation of voltage-gated sodium channels. This inactivation prevents the channel from opening, helping end the action potential. Lacosamide slows the recovery from inactivation and hence reduce the ability of neurons to fire action potentials. Inactivation only occurs in neurons firing action potentials; this means that drugs that modulate fast inactivation selectively reduce the firing in active cells. Slow inactivation is similar but does not produce complete blockade of voltage gated sodium channels, with both activation and inactivation occurring over hundreds of milliseconds or more. Lacosamide makes this inactivation happen at less depolarized membrane potentials. This means that lacosamide only affects neurons which are depolarized or active for long periods of time, typical of neurons at the focus of epilepsy (“The investigational anticonvulsant lacosamide selectively enhances slow inactivation of voltage-gated sodium channels” Errington et al., 2008, Molecular Pharmacology, 73(1): 157-69). Lacosamide administration results in the inhibition of repetitive neuronal firing, the stabilization of hyperexcitable neuronal membranes, and the reduction of long-term channel availability, but does not affect physiological function (“Development of lacosamide for the treatment of partial-onset seizures” Doty et al., 2013, Ann N Y Acad Sci. 1291: 56-68). Lacosamide does not affect α-amino-3-hydroxy-5-methyl-4-isoxazolepropionic acid (AMPA), kainate, N-methyl-D-aspartate (NMDA), GABAA, GABAB or a variety of dopaminergic, serotonergic, adrenergic, muscarinic or cannabinoid receptors and does not block potassium or calcium currents (“Seeking a mechanism of action for the novel anticonvulsant lacosamide” Errington et al., 2006, Neuropharmacology, 50(8): 1016-29). Lacosamide does not modulate the reuptake of neurotransmitters including norepinephrine, dopamine, and serotonin (“Lacosamide: a review of preclinical properties” Beyreuther et al., 2007, CNS Drug Reviews, 13 (1): 21-42). In addition, it does not inhibit GABA transaminase. Unlike many other non-stimulant compounds, such as phenytoin, carbamazepine, oxacarbamazepine and lamotrigine, lacosamide has been reported not to inhibit voltage-gated calcium channels (L-, N-, P/Q, T-type channels) (“Voltage-gated calcium channels are not affected by the novel anti-epileptic drug lacosamide.” Wang. and Khanna, 2011, Translational neuroscience, 2(1): 13-22).

Therefore, the agent contemplated herein preferably does not (or not significantly) affect voltage-gated calcium channels, preferably it does not (or not significantly) inhibit voltage-gated calcium channels.

Also contemplated are analogues of lacosamide, wherein the analogues are functionally equivalent to lacosamide. The analogues may have significant structural similarity to the structure of lacosamide. By “functionally equivalent” is meant that the analogue is a non-stimulant; preferably does not (or not significantly) affect voltage-gated calcium channels; and/or and has a therapeutic effect on ADD and/or ADHD patients that is equivalent to that of lacosamide. The analogue may, for example, be the S enantiomer, (S)-2-(acetylamino)-N-benzyl-3-methoxypropanamide; a racemic mixture of the R enantiomer and the S enantiomer; or rufinamide. Rufinamide's chemical name is 1-[(2,6-difluorophenyl)methyl]-1H-1,2,3-triazole-4 carboxamide (marketed under brand name BANZEL™ or Inovelon™) Similarly to lacosamide, rufinamide has been shown to act through voltage-gated sodium channels (White et al. The anticonvulsant profile of rufinamide (CGP 33101) in rodent seizure models. Epilepsia 49(7):1213-1220, 2008).

The present inventor has surprisingly discovered that non-stimulant compounds such as lacosamide can effectively treat and manage ADHD symptoms in a subject suffering from ADHD, such as combined type ADHD.

Thus, in a first aspect, the invention provides a method for treating attention deficit disorder (ADD) or attention deficit hyperactivity disorder (ADHD) comprising, consisting essentially of or consisting of administering a therapeutically active amount of an agent to a subject suffering from ADD or ADHD, wherein the agent is active in the brain but does not stimulate neurotransmitter release or recapture transporters (in the brain).

In a related aspect, the invention also provides an agent for use in a method for treating attention deficit disorder (ADD) or attention deficit hyperactivity disorder (ADHD), the method comprising, consisting essentially of or consisting of administering a therapeutically active amount of the agent to a subject suffering from ADD or ADHD, wherein the agent is active in the brain but does not stimulate neurotransmitter release or recapture transporters (in the brain).

In a further related aspect, the invention also provides use of an agent in the manufacture of a medicament for treating attention deficit disorder (ADD) or attention deficit hyperactivity disorder (ADHD), the method of treatment comprising, consisting essentially of or consisting of administering a therapeutically active amount of the agent to a subject suffering from ADD or ADHD, wherein the agent is active in the brain but does not stimulate neurotransmitter release or recapture transporters (in the brain).

In particular embodiments of any of these aspects, the subject does not also suffer from epilepsy and/or bipolar disorder. In some embodiments, the subject does not also suffer from any seizures and/or neuropathic pain.

In particular embodiments of any of these aspects, the subject suffering from ADD or ADHD may have one or more comorbidities, which may, for example, include epilepsy, bipolar disorder, seizures and/or neuropathic pain. In accordance with the invention, lacosamide or an analogue thereof is used to treat the ADD or ADHD irrespective of the presence or absence of any comorbidities (and irrespective of any treatment therefor the patient may be receiving). Thus, where the patient suffers from a comorbidity such as epilepsy, the purpose of the administration in accordance with the invention of the agent such as lacosamide or an analogue thereof is to treat the ADD or ADHD; it is not to treat any comorbid conditions such as epilepsy.

The term “agent” is used to mean a substance such as a compound. By “active in the brain” it is meant that the agent can interact directly with the brain, for instance by enhancing the slow inactivation of voltage-sensitive sodium channels in the brain thereby stabilizing neuronal membranes. Crucially, according to all aspects of the invention, the agent is a “non-stimulant”, that is to say it does not stimulate neurotransmitter release or recapture transporters (in the brain).

As used herein, “administering” means introducing the agent (e.g. a compound, preferably lacosamide or a functionally equivalent analogue thereof) into the subject's body as described in more detail below. Examples include but are not limited to oral, topical, buccal, sublingual, pulmonary, transdermal, transmucosal, as well as subcutaneous, intraperitoneal, intravenous, and intramuscular injection or in the form of liquid or solid doses via the alimentary canal. Oral delivery is preferred.

As used herein, the phrase “a therapeutically active amount” means an amount of an agent (e.g. a compound, preferably lacosamide or a functionally equivalent analogue thereof) that, when administered to a subject for treating ADD/ADHD, is sufficient to effect such treatment of ADD/ADHD. A “therapeutically active amount” will vary depending, for instance, on factors such as the specific agent used, the severity of subject's ADD/ADHD, the age and relative health of the subject and the route and form of administration. Determining the relevant therapeutically active amount for a specific subject based on such factors is routine for the person skilled in the art (e.g. an attending medical practitioner). Treatment of ADD/ADHD as described herein should be understood to mean an improvement in one of more of the symptoms. This may include, for instance, an improvement in focus, concentration and/or memory, being more organised and/or being able to complete tasks more easily. It may also include a reduction in hyperactivity, fidgetiness and/or restlessness and/or a reduction in immature and/or destructive behaviours. However, it will be understood by the skilled person based on the teachings herein that treatment is not necessarily intended to mean a cure for ADD/ADHD in view of the fact that it is a neurological syndrome. Instead, treatment generally refers to an improvement in one or more of the symptoms as described, typically a progressive and long-lasting improvement.

In a preferred embodiment of the agent is lacosamide or a functionally equivalent analogue thereof such as the S-enantiomer, the S and R racemate, or rufinamide; preferably it is lacosamide.

Thus, in another aspect, the invention provides a method for treating attention deficit disorder (ADD) or attention deficit hyperactivity disorder (ADHD) comprising, consisting essentially of or consisting of administering a therapeutically active amount of lacosamide or a functionally equivalent analogue thereof to a subject suffering from ADD or ADHD.

In a related aspect, the invention also provides an agent for use in a method for treating attention deficit disorder (ADD) or attention deficit hyperactivity disorder (ADHD), the method comprising, consisting essentially of or consisting of administering a therapeutically active amount of the agent to a subject suffering from ADD or ADHD, wherein the agent is lacosamide or a functionally equivalent analogue thereof.

In a further related aspect, the invention provides the use of an agent in the manufacture of a medicament for treating attention deficit disorder (ADD) or attention deficit hyperactivity disorder (ADHD), the method of treatment comprising, consisting essentially of or consisting of administering a therapeutically active amount of the agent to a subject suffering from ADD or ADHD, wherein the agent is lacosamide or a functionally equivalent analogue thereof.

In particular embodiments, the subject suffers from ADHD, such as the combined type ADHD.

In particular embodiments, the subject does not also suffer from epilepsy or bipolar disorder. In some embodiments, the subject does not also suffer from any seizures and/or neuropathic pain.

As shown in the Examples, treatment of subjects suffering from ADD or ADHD with lacosamide eliminated, alleviated or ameliorated certain symptoms of ADD or ADHD. For example, overabundant thought and disordered thought were alleviated or eliminated; and sleep activity was improved.

It will be appreciated that the treatment of ADD or ADHD may result in the elimination, alleviation or amelioration of one or more symptoms of ADD or ADHD and need not necessarily alleviate or ameliorate every symptom of ADD or ADHD that the patient experienced prior to treatment. The treatment may also result in an increase in the subject's IQ.

By ‘elimination’ of a symptom is meant that the subject essentially no longer experiences that symptom. By ‘alleviation’ of a symptom is meant that the subject experiences a significant improvement in the symptom. The alleviation is preferably from severe to mild. By ‘amelioration’ of a symptom is meant that the subject experiences a modest improvement in the symptom.

Thus, in particular embodiments, the treatment, use and the like provided herein may result in the elimination, alleviation or amelioration of one or more symptoms of ADD or ADHD. For example, one or more symptoms may be alleviated and one or more different symptoms may be ameliorated. One or more of the following, or one or more symptoms within these categories, may be eliminated, alleviated or ameliorated:

(i) impaired cognitive function;

(ii) impaired cognitive performance;

(iii) hyperactivity;

(iv) impaired circadian rhythm and/or sleep activity, such as difficulty with falling asleep, sleep interruptions, waking up tired, and/or unrealistic dreams; and/or

(v) impaired mood.

More particularly, one or more of the following may be eliminated, alleviated or ameliorated: Overabundant thought; Disordered thought; Tachypsychia; Hypercritical thinking; blocking thoughts; sudden disconnections; distressing thoughts; mood swings; hypomania; lethargy; tiredness; and/or impaired non-verbal reasoning.

Thus, alternatively viewed, provided is a method of eliminating, alleviating and/or ameliorating one or more of the above symptoms by administering a therapeutically effective amount of an agent as defined herein, preferably lacosamide or a functionally equivalent analogue thereof, to a subject in need thereof. The subject may suffer from ADD or ADHD.

In some embodiments, according to all aspects of the invention and preferably in the case of lacosamide or a functionally equivalent analogue thereof, the therapeutically active amount may be in the range of about 1-12 milligrams of the agent per kilogram body weight of the subject per day (mg/kg/day). In specific embodiments, the therapeutically active amount may be at least or about 1 mg/kg/day, at least or about 2 mg/kg/day, at least or about 4 mg/kg/day, at least or about 6 mg/kg/day, at least or about 8 mg/kg/day, or at least or about 10 mg/kg/day. In further embodiments, the therapeutically active amount may be in the range of about 2-6 mg/kg/day, 1-10 mg/kg/day or 1-15 mg/kg/day.

In further embodiments, the therapeutically active amount of the agent (e.g. lacosamide) is administered per the following treatment regimen:

an initial amount of 2 mg/kg/day, which if tolerated by the patient is followed by:

(ii) every 1-2 weeks increasing the amount by 2 mg/kg/day up to a maximum amount in the range 8-12 mg/kg/day and maintaining the daily amount thereat (i.e. the maintenance amount).

In further embodiments, the therapeutically active amount of the agent (e.g. lacosamide) is administered per the following treatment regimen:
(i) 2 mg/kg/day for 1-2 weeks, followed by:
(ii) 4 mg/kg/day for 1-2 weeks, followed by:
(iii) every 1-2 weeks increasing the amount by 2 mg/kg/day or less up to a maximum amount in the range 8-12 mg/kg/day and maintaining the daily amount thereat (i.e. the maintenance amount).

The exact maintenance amount, according to all aspects of the invention, can be routinely determined by the skilled person based on the specific subject's physical condition, severity of ADD/ADHD symptoms and tolerance of the agent being administered. Generally speaking, the exact maintenance amount will be a compromise between improvement of the ADD/ADHD symptoms against the physiological tolerance of the specific subject for that exact amount (e.g. the observance/potential for unwanted side effects). This is specific to each individual subject and it is routine for the skilled person in the art (e.g. a medical practitioner) to determine the exact maintenance amount for a specific subject based on the aforementioned factors.

In some embodiments, according to all aspects of the invention and preferably in the case of lacosamide, the therapeutically active amount may be in the range of about 25-600 or 50-400 mg/day. For instance, it may be at least or about 25, 50, 75, 100, 125, 150, 175, 200, 225, 250,275, 300, 325, 350, 375, 400, 425, 450, 475, 500, 525, 550, 575 or 600 mg/day. Typically, it is at least 100 mg/day.

Whilst the amount to be administered may be dependent on the weight (in kilograms) of the subject, this is not essential. Thus, in some embodiments, the therapeutically active amount of the agent (e.g. lacosamide) is 50-100 mg/day, preferably at least 100 mg/day. In further embodiments, the amount of agent may be incrementally increased over time from a starting amount to a maintenance amount. In some embodiments, the amount of agent is incrementally increased over time from a starting amount to a maintenance amount. For instance, in particular embodiments, the therapeutically active amount of the agent (e.g. lacosamide) is administered per the following treatment regimen:

(i) an initial amount of 50-100 mg/day, which if tolerated by the patient is followed by:

(ii) every 1-2 weeks increasing the amount by 50-100 mg up to a maximum amount in the range 200-600 mg/day and maintaining the daily amount thereat (i.e. the maintenance amount).

In further embodiments, the therapeutically active amount of the agent (e.g. lacosamide) is administered per the following treatment regimen:

(i) an initial amount of 50 mg/day, which if tolerated by the patient is followed by:

(ii) every 1-2 weeks increasing the amount by 50 mg up to a maximum amount in the range 200-300 mg/day and maintaining the daily amount thereat (i.e. the maintenance amount).

For example, in a subject weighing less than 50 kg, the treatment regimen may be:
(i) 50 mg/day for the first 1-2 weeks, followed by:
(ii) 100 mg/day for the next 1-2 weeks, followed by:
(iii) 150 mg/day for the next 1-2 weeks.

After 3-6 weeks, if the skilled person (e.g. medical practitioner) determines that a higher dose is needed to (further) improve the subject's ADD/ADHD symptoms, the agent (e.g. lacosamide) may be administered at:
(iv) 200 mg/day for 1-2 weeks;

and so on up to a maximum amount of no more than 250 mg/day depending on the amount needed to treat the subject's symptoms, and the subject's tolerance of the agent.

In particular example, in a subject weighing less than 50 kg, the treatment regimen may be:
(i) 50 mg/day for 1-2 weeks, followed by:
(ii) 100 mg/day for 1-2 weeks, followed by:
(iii) 150 mg/day for 1-2 weeks, followed by:
(iv) 200 mg/day for 1-2 weeks, followed by:
(v) 250 mg/day for 1-2 weeks;
(vi) maintaining the amount at 250 mg/day.

In further embodiments, the therapeutically active amount of the agent (e.g. lacosamide) is administered per the following treatment regimen:
(i) an initial amount of 100 mg/day, which if tolerated by the patient is followed by:
(ii) every 1-2 weeks increasing the amount by 100 mg up to a maximum amount in the range 400-600 mg/day and maintaining the daily amount thereat (i.e. the maintenance amount).

For example, in a subject weighting 50 kg or more, the treatment regimen may be:
(i) 100 mg/day for the first 1-2 weeks, followed by:
(ii) 200 mg/day for the next 1-2 weeks, followed by:
(iii) 300 mg/day for the next 1-2 weeks, followed by:
(iv) 400 mg/day for the next 1-2 weeks.

After 4-8 weeks, if the skilled person (e.g. medical practitioner) determines that a higher dose is needed to (further) improve the subject's ADD/ADHD symptoms, the agent (e.g. lacosamide) may be administered at:
(v) 500 mg/day for 1-2 weeks;

and so on up to a maximum amount of no more than 600 mg/day depending on the amount needed to treat the subject's symptoms, and the subject's tolerance of the agent.

In some embodiments, the therapeutically active amount is 250 mg/day or less, for instance 25, 50, 75, 100, 125, 150, 175, 200, or 225 mg/day.

In particular embodiments, the agent, such as lacosamide, may be administered as a single dose once per day. In preferred embodiments, it may be administered as two, three or more separate doses throughout the day, preferably two. That is to say, the therapeutically active amount per day is divided into two, three or more doses administered to the subject separately throughout the day. For example, a therapeutically active amount of 150 mg/day may be administered as two 75 mg doses per day or three 50 mg doses per day. The multiple doses per day do not necessarily need to be the same amount. For instance, a therapeutically active amount of 150 mg/day may be administered as one 100 mg dose later followed by a 50 mg dose. The skilled person (e.g. a medical practitioner) is well able to determine an appropriate dosage regimen for the subject according to the subject's specific circumstances.

The treatment (administration of the agent, such as lacosamide, on a regular basis, such as daily) may be carried out for a suitable period of time, such as at least 2, 3, 4, 5, 6, 7, 8, 9 or 10 weeks, months or years. If it is well tolerated, the treatment may be carried out indefinitely. Preferably, treatment is carried out for a minimum of about 2 months.

In particular embodiments, the subject suffers from no other neurological comorbidities (disorders) i.e. disorders of the nervous system, particularly the brain. That is to say, the subject suffers from ADD/ADHD but does not suffer from any other neurological comorbidity (disorder) such as epilepsy, bipolar disorder and the like. However, in some embodiments, the subject may suffer from autism.

According to all aspects of the invention, the subject is preferably not treated with a “stimulant” agent immediately prior to, during, and/or after the treatment regime of the present invention, although in some embodiments the subject may previously have received treatment with a “stimulant” agent, which treatment should preferably be discontinued prior to treatment according to the present invention. In preferred embodiments, the “non-stimulant” agent (e.g. lacosamide) is administered as a monotherapy.

According to all aspects of the invention, the subject is a mammal, preferably a human. In particular embodiments, the subject is a child (i.e. one who is growing/maturing physiologically and neurologically towards an adult phenotype). For instance, a “child” may be considered to be a pre-pubescent subject or one who is in the process of completing puberty. Particularly when the subject is a human, a “child” may also be considered to be an individual under the age of 18 in some cases. In preferred embodiments, the child is between 2-12 or at least 2, 3, 4, 5, 6, 7 or 8 and no more than 18, 17 or 16 years old. In more preferred embodiments, the child is 10 years old or less. Thus, the subject may, for example, weigh less than or about 60, 50, 40 or 30 kg. The invention is, however, also applied to adults in some embodiments, in which case the subject may, for example, weigh more than or about 50, 60, 70, 80, 90, 100, or 110 kg.

According to all aspects of the invention, the agent, which is preferably lacosamide, may be formulated as a pharmaceutical composition with one or more pharmaceutically acceptable additives/solvents. For instance, the agent may be formulated as pills, tablets or capsules combined with one or more pharmaceutically acceptable solid carriers or as a solution in one or more pharmaceutically acceptable solvents, or as an emulsion, suspension or dispersion in one or more pharmaceutically acceptable solvents or carriers. In some embodiments, the formulation may also include other pharmaceutically acceptable excipients such as stabilizers, anti-oxidants, binders, colouring agents or emulsifying or taste-modifying agents and extended release formulations. In some embodiments, the agent may be formulated as a pharmaceutically acceptable salt, ester, prodrug or metabolite, such as a pharmaceutically acceptable salt, ester, prodrug or metabolite of lacosamide.

According to all aspects of the invention, the agent may be administered orally, topically, parenterally or transdermally or by inhalation. The agent may be administered by injection or intravenous infusion using suitable sterile solutions. Topical dosage forms may be creams, ointments, patches, or similar vehicles suitable for transdermal and topical dosage forms. Oral administration is preferred. For instance, in the case of lacosamide, in preferred embodiments it is administered as a film-coated tablet or an oral solution (e.g. under the brand name VIMPAT®) but intravenous administration is also possible.

As used herein, “oral dosage” forms may include capsules (i.e., a solid oral dosage form consisting of a shell and a filling), whereby the shell is composed of a single sealed enclosure, or two halves that fit together and which are sometimes sealed with a band, and whereby capsule shells may be made from gelatin, starch, or cellulose, or other suitable materials, may be soft or hard, and are filled with a solid or liquid ingredients that can be poured or squeezed. The oral dosage form may also be a capsule or coated pellets, in which the agent is enclosed within either a hard or soft soluble container or “shell” made from a suitable form of gelatin. The agent itself may be in the form of granules to which varying amount of coating have been applied or in a capsule coated extended release, in which the agent is enclosed within either a hard or soft soluble container or “shell” made from a suitable form of gelatin. Additionally, the capsule may be covered in a designated coating which releases the agent in such a manner to allow at least a reduction in dosing frequency as compared to that drug or drugs presented as a conventional dosage form.

The oral dosage form may further be a capsule delayed release, in which the agent is enclosed within either a hard or soft soluble container made from a suitable form of gelatin, and which releases the agent at a time other than promptly after administration, whereby enteric-coated articles are delayed release dosage forms. Capsule delayed release pellets, in which the agent is enclosed within either a hard or soft container or “shell” are also useful. In these cases, the agent itself is in the form of granules to which enteric coating has been applied, thus delaying release of the agent until its passing into the intestine. Capsule extended release and capsule film-coated extended release are also useful.

Additionally, the capsule may be covered in a designated film coating which releases the agent in such a manner to allow at least a reduction in dosing frequency as compared to that agent presented as a conventional dosage form. Examples include capsule gelatin coated (a solid dosage form in which the agent is enclosed within either a hard or soft soluble container made from a suitable form of gelatin; through a banding process, the capsule is coated with additional layers of gelatin so as to form a complete seal) and capsule liquid filled (a solid dosage form in which the agent is enclosed within a soluble, gelatin shell which is plasticized by the addition of a polyol, such as sorbitol or glycerin, and is therefore of a somewhat thicker consistency than that of a hard shell capsule).

In some embodiments, the agent may be dissolved or suspended in a liquid vehicle or formulated as a granule (a small particle or grain), a pellet (a small sterile solid mass consisting of a highly purified agent, with or without excipients, made by the formation of granules, or by compression and moulding), or a pellet coated extended release (a solid dosage form in which the agent itself is in the form of granules to which varying amounts of coating have been applied, and which releases the agent in such a manner to allow a reduction in dosing frequency as compared to that agent presented as a conventional dosage form).

Other forms include pills (a small, round solid dosage form containing the agent intended for oral administration), powder (an intimate mixture of dry, finely divided agent with one or more pharmaceutically acceptable additives that may be intended for internal or external use), elixir (a clear, pleasantly flavored, sweetened hydroalcoholic liquid containing dissolved agent; it is intended for oral use), chewing gum (a sweetened and flavored insoluble plastic material of various shapes which when chewed, releases the agent substance into the oral cavity), syrup (an oral solution containing the agent and high concentrations of sucrose or other sugars; the term has also been used to include any other liquid dosage form prepared in a sweet and viscid vehicle, including oral suspensions), tablet (a solid dosage form containing the agent with or without suitable diluents), tablet chewable (a solid dosage form containing the agent with or without suitable diluents that is intended to be chewed, producing a pleasant tasting residue in the oral cavity that is easily swallowed and does not leave a bitter or unpleasant after-taste), tablet coated or tablet delayed release, tablet dispersible, tablet effervescent, tablet extended release, tablet film coated, or tablet film coated extended release where the tablet is formulated in such manner as to make the contained agent available over an extended period of time following ingestion.

In other forms, a tablet for solution, tablet for suspension, tablet multilayer, tablet multilayer extended release may be provided, where the tablet is formulated in such manner as to allow at least a reduction in dosing frequency as compared to that agent presented as a conventional dosage form. A tablet orally disintegrating, tablet orally disintegrating delayed release, tablet soluble, tablet sugar coated, osmotic, and the like are also suitable.

The oral dosage form composition may contain, in addition to the agent, one or more inactive pharmaceutical ingredients such as diluents, solubilizers, alcohols, binders, controlled release polymers, enteric polymers, disintegrants, excipients, colorants, flavorants, sweeteners, antioxidants, preservatives, pigments, additives, fillers, suspension agents, surfactants (e.g., anionic, cationic, amphoteric and nonionic), and the like. Various FDA-approved topical inactive ingredients are found at the FDA's “The Inactive Ingredients Database” that contains inactive ingredients specifically intended as such by the manufacturer. For instance, in the case of lacosamide, in preferred embodiments it is formulated as a film-coated tablet or oral solution (e.g. under the brand name VIMPAT®). In addition to lacosamide, the tablets may comprise one or more or all of colloidal silicon dioxide, crospovidone, hydroxypropylcellulose, magnesium stearate, microcrystalline cellulose, polyethylene glycol, polyvinyl alcohol, talc, titanium dioxide, and dye pigments (50 mg tablets: red iron oxide, black iron oxide, FD&C Blue No. 2/indigo carmine aluminum lake; 100 mg tablets: yellow iron oxide; 150 mg tablets: yellow iron oxide, red iron oxide, black iron oxide; 200 mg tablets: FD&C Blue No. 2/indigo carmine aluminum lake). In addition to lacosamide, the oral solution may comprise one or more or all of purified water, sorbitol solution, glycerin, polyethylene glycol, carboxymethylcellulose sodium, acesulfame potassium, methylparaben, flavoring (including natural and artificial flavors, propylene glycol, aspartame, and maltol), anhydrous citric acid and sodium chloride.

As used herein, injectable and infusion dosage forms include, but are not limited to, a liposomal injectable, which either consists of or forms liposomes (a lipid bilayer vesicle usually composed of phospholipids which is used to encapsulate the agent); an injection, which includes a sterile preparation intended for parenteral use; an emulsion injection, which includes an emulsion consisting of a sterile, pyrogen-free preparation intended to be administered parenterally; or a lipid complex injection. For instance, in the case of lacosamide, in some embodiments it is formulated as a solution for intravenous administration (e.g. under the brand name VIMPAT®).

Other forms include a powder for solution injection, which is a sterile preparation intended for reconstitution to form a solution for parenteral use; a powder for suspension injection that is a sterile preparation intended for reconstitution to form a suspension for parenteral use; a powder lyophilized for liposomal suspension injection, which is a sterile freeze dried preparation intended for reconstitution for parenteral use which has been formulated in a manner that would allow liposomes (a lipid bilayer vesicle usually composed of phospholipids which is used to encapsulate the agent, either within a lipid bilayer or in an aqueous space) to be formed upon reconstitution; or a powder lyophilized for solution injection, wherein lyophilization (“freeze drying”) is a process which involves the removal of water from products in the frozen state at extremely low pressures.

As used herein, a “solution injection” refers to a liquid preparation containing one or more agents dissolved in a suitable solvent or mixture of mutually miscible solvents that is suitable for injection. A “solution concentrate injection” refers to a sterile preparation for parenteral use which, upon the addition of suitable solvents, yields a solution conforming in all respects to the requirements for injections.

A suspension injection comprises a liquid preparation, suitable for injection, which consists of solid particles dispersed throughout a liquid phase in which the particles are not soluble that can also consist of an oil phase dispersed throughout an aqueous phase, or vice-versa. A suspension liposomal injection comprises a liquid preparation, suitable for injection, which consists of an oil phase dispersed throughout an aqueous phase in such a manner that liposomes (a lipid bilayer vesicle usually composed of phospholipids which is used to encapsulate the agent, either within a lipid bilayer or in an aqueous space) are formed. A suspension sonicated injection comprises a liquid preparation, suitable for injection, which consists of solid particles dispersed throughout a liquid phase in which the particles are not soluble. In addition, the product is sonicated while a gas is bubbled through the suspension, and this results in the formation of microspheres by the solid particles.

The parenteral carrier system includes one or more pharmaceutically suitable excipients, such as solvents and co-solvents, solubilizing agents, wetting agents, suspending agents, thickening agents, emulsifying agents, chelating agents, buffers, pH adjusters, antioxidants, reducing agents, antimicrobial preservatives, bulking agents, protectants, tonicity adjusters, and special additives. Formulations suitable for parenteral administration conveniently comprise a sterile oily or aqueous preparation of the agent which is preferably isotonic with the blood of the recipient but this is not essential. For instance, in the case of lacosamide formulated as a solution for intravenous administration (e.g. under the brand name VIMPAT®), the solution may additionally comprise one or more or all of sodium chloride, water and hydrochloric acid (for pH adjustment to a pH between 3.5-5.0).

As used herein, inhalation dosage forms include, but are not limited to, an aerosol (a product that is packaged under pressure and contains the agent which is released upon activation of an appropriate valve system intended for topical application to the skin as well as local application into the nose (nasal aerosols), mouth (lingual and sublingual aerosols), or lungs (inhalation aerosols)). A foam aerosol is a dosage form containing the agent, surfactants, aqueous or nonaqueous liquids, and propellants, whereby if the propellant is in the internal (discontinuous) phase (i.e., of the oil-in-water type), a stable foam is discharged, and if the propellant is in the external (continuous) phase (i.e., of the water-in-oil type), a spray or a quick-breaking foam is discharged. A metered aerosol is a pressurized dosage form consisting of metered dose valves which allow for the delivery of a uniform quantity of spray upon each activation. A powder aerosol is a product that is packaged under pressure and contains the agent, in the form of a powder, that is released upon activation of an appropriate valve system. An aerosol spray is an aerosol product which utilizes a compressed gas as the propellant to provide the force necessary to expel the product as a wet spray and being applicable to solutions of the agent in aqueous solvent(s).

As used herein, transdermal dosage form includes, but is not limited to, a patch (a drug delivery system that often contains an adhesive backing that is usually applied to an external site on the body, whereby the ingredients (including the agent) either passively diffuse from, or are actively transported from, some portion of the patch, and whereby depending upon the patch, the ingredients (including the agent) are either delivered to the outer surface of the body or into the body. Various types of transdermal patches such as matrix, reservoir and others are known in the art.

As used herein, the topical dosage form includes various dosage forms known in the art such as lotions (an emulsion, liquid dosage form, whereby this dosage form is generally for external application to the skin), lotion augmented (a lotion dosage form that enhances agent delivery, whereby augmentation does not refer to the strength of the agent in the dosage form), gels (a semisolid dosage form that contains a gelling agent to provide stiffness to a solution or a colloidal dispersion, whereby the gel may contain suspended particles) and ointments (a semisolid dosage form, usually containing less than 20% water and volatiles and greater than 50% hydrocarbons, waxes, or polyols as the vehicle, whereby this dosage form is generally for external application to the skin or mucous membranes). Further embodiments include ointment augmented (an ointment dosage form that enhances agent delivery, whereby augmentation does not refer to the strength of the agent in the dosage form), creams (an emulsion, semisolid dosage form, usually containing greater than 20% water and volatiles and/or less than 50% hydrocarbons, waxes, or polyols may also be used as the vehicle, whereby this dosage form is generally for external application to the skin or mucous membranes) and cream augmented (a cream dosage form that enhances agent delivery, whereby augmentation does not refer to the strength of the agent in the dosage form). As used herein, an “emulsion” refers to a dosage form consisting of a two-phase system comprised of at least two immiscible liquids, one of which is dispersed as droplets, internal or dispersed phase, within the other liquid, external or continuous phase, generally stabilized with one or more emulsifying agents, whereby emulsion is used as a dosage form term unless a more specific term is applicable (e.g. cream, lotion, ointment). Further embodiments include suspensions (a liquid dosage form that contains solid particles dispersed in a liquid vehicle), suspension extended release, pastes (a semisolid dosage form, containing a large proportion, 20-50%, of solids finely dispersed in a fatty vehicle, whereby this dosage form is generally for external application to the skin or mucous membranes), solutions (a clear, homogeneous liquid dosage form that contains one or more chemical substances dissolved in a solvent or mixture of mutually miscible solvents), and powders.

The topical dosage form composition contains the agent and one or more inactive pharmaceutical ingredients such as excipients, colorants, pigments, additives, fillers, emollients, surfactants (e.g., anionic, cationic, amphoteric and nonionic), penetration enhancers (e.g., alcohols, fatty alcohols, fatty acids, fatty acid esters and polyols), and the like. Various FDA-approved topical inactive ingredients are found at the FDA's “The Inactive Ingredients Database” that contains inactive ingredients specifically intended as such by the manufacturer.

As shown in the Examples, lacosamide is slow acting, resulting in a gradual improvement of symptoms. Without wishing to be bound by theory, it is believed that the agent contemplated herein, particularly lacosamide, acts in an intracellular manner.

These and other aspects of the invention will now be described with reference to the accompanying Figures, in which:

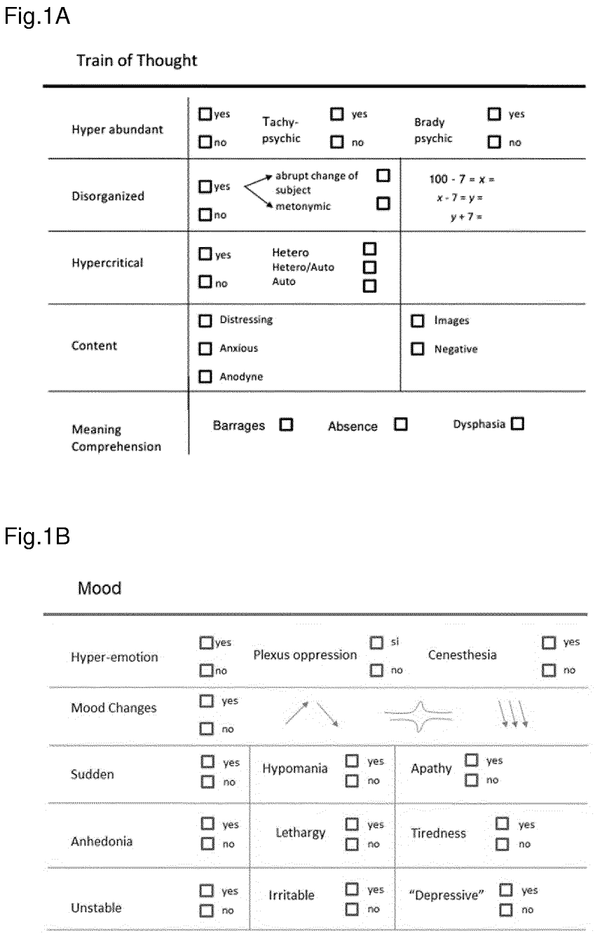

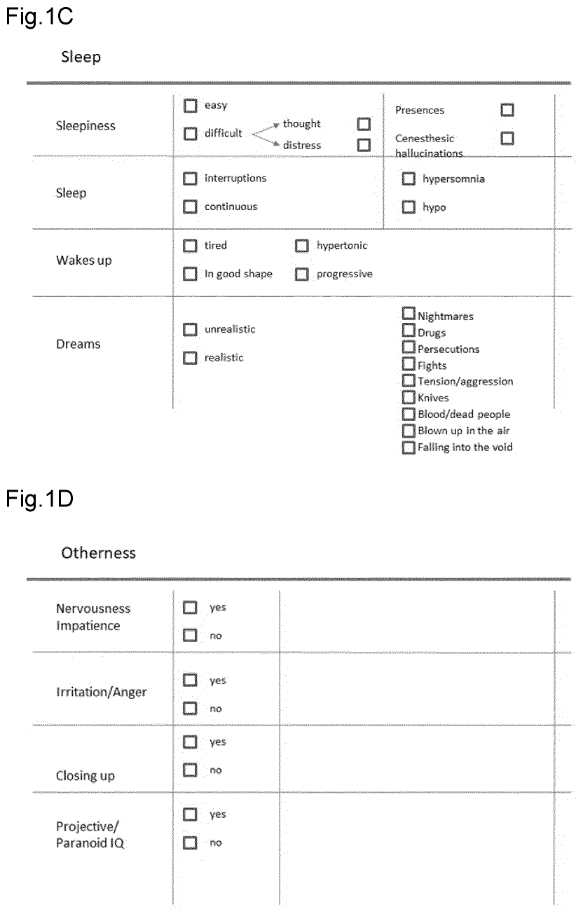

FIG. 1A-D shows questionnaire templates for Train of Thought, Mood, Sleep and Otherness evaluations, respectively.

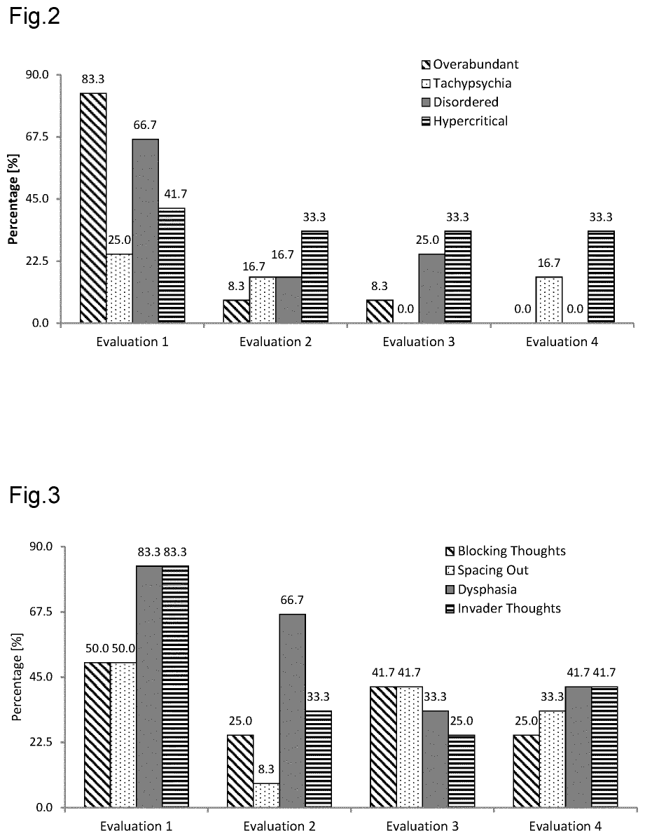

FIG. 2 shows the results of four evaluations of the Trend of Thought in patients treated with lacosamide.

FIG. 3 shows the results of four evaluations of the Cognitive Thinking in patients treated with lacosamide.

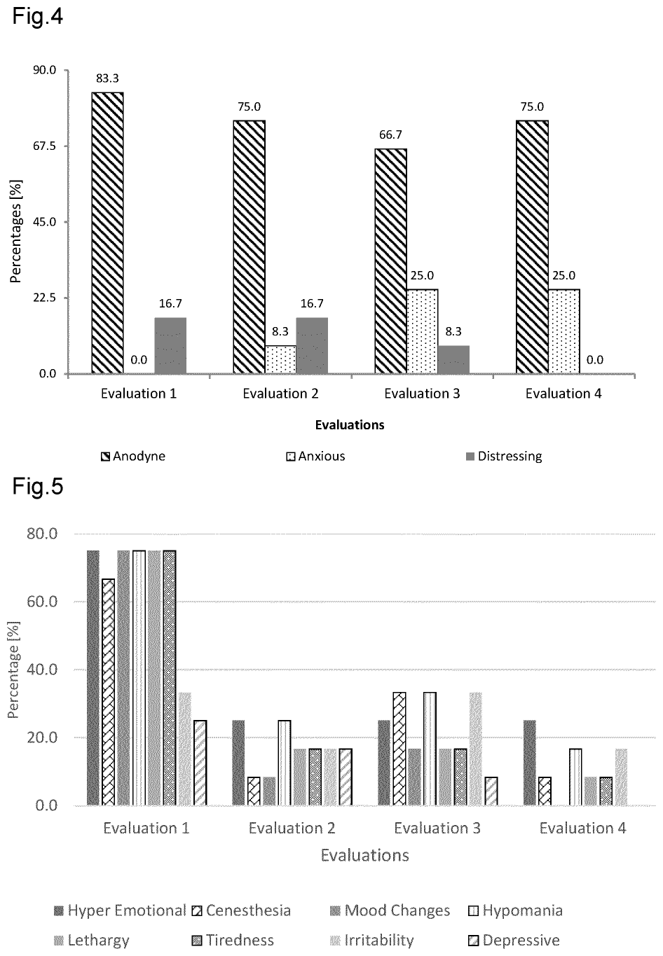

FIG. 4 shows the results of four evaluations of the Thought Content in patients treated with lacosamide.

FIG. 5 shows the results of four evaluations of the Mood and Neuro-vegetative Function in patients treated with lacosamide.

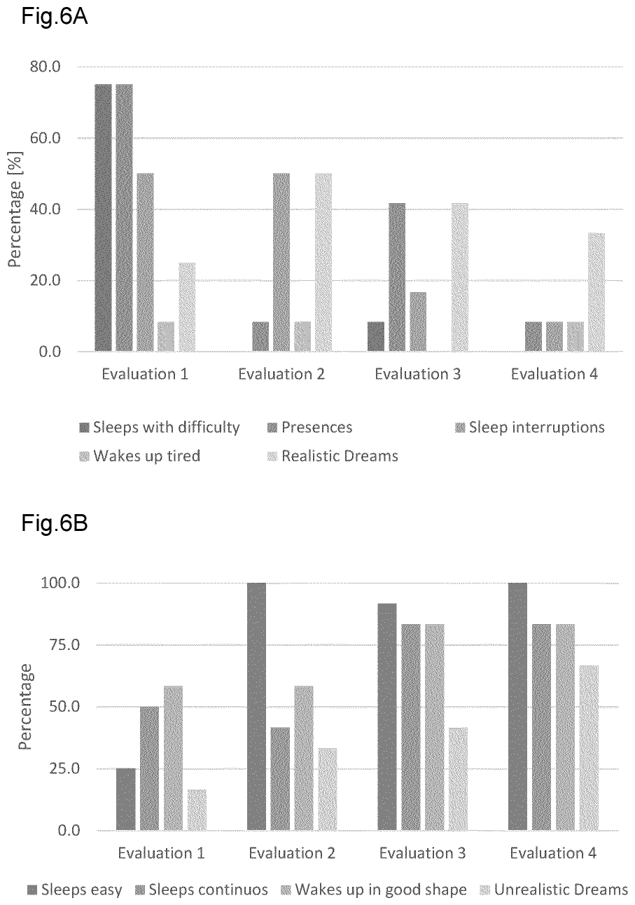

FIGS. 6A and 6B show the results of four evaluations of the Sleep Activity in patients treated with lacosamide.

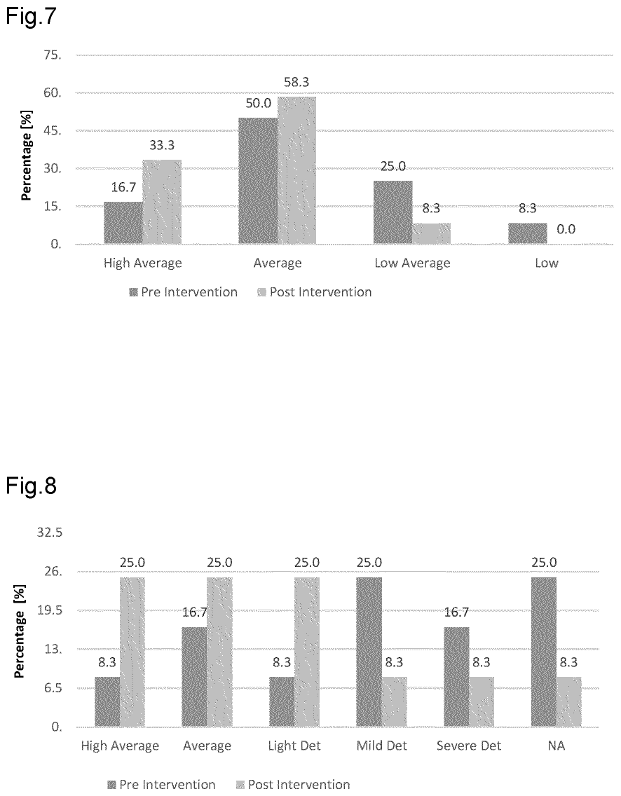

FIG. 7 shows the results of the TONI-2 test in patients treated with lacosamide.

FIG. 8 shows the results of the WCST Flexibility test in patients treated with lacosamide.

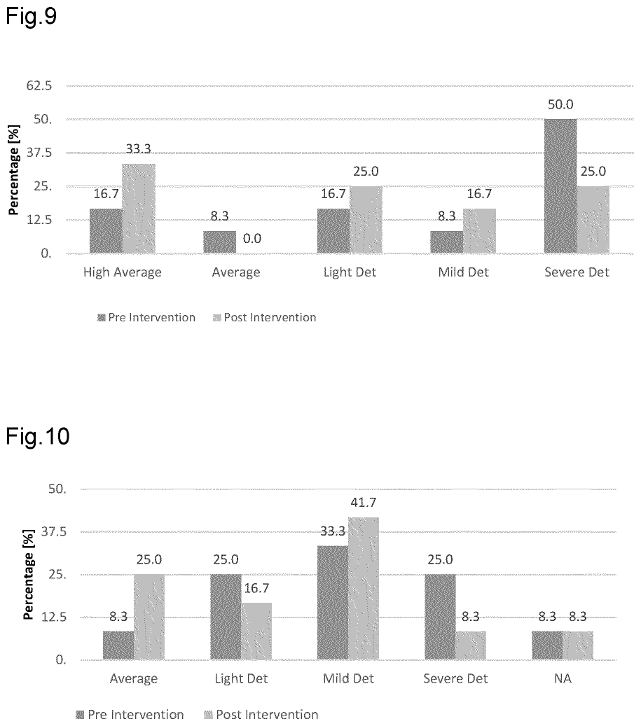

FIG. 9 shows the results of the WCST Reasoning test in patients treated with lacosamide.

FIG. 10 shows the results of the STROOP-P test in patients treated with lacosamide.

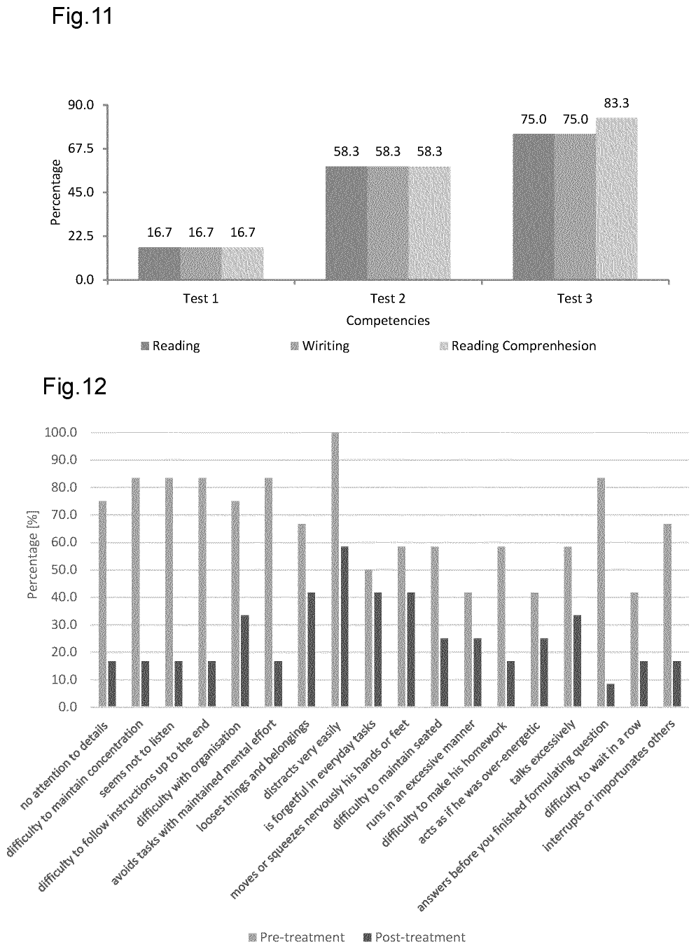

FIG. 11 shows the results of three evaluations of Spanish Language in patients treated with lacosamide.

FIG. 12 shows the results of two Tutor's evaluations of the Behaviour in patients treated with lacosamide.

The Invention is further disclosed in the following clauses:

1. A method for treating attention deficit disorder (ADD) or attention deficit hyperactivity disorder (ADHD) comprising administering a therapeutically active amount of an agent to a subject suffering from ADD or ADHD, wherein the agent is active in the brain but does not stimulate neurotransmitter release or recapture transporters (in the brain).

2. The method of clause 1 wherein the subject does not also suffer from epilepsy, bipolar disorder, any seizures, and/or neuropathic pain.

3. The method of clause 1 or 2 wherein the subject suffers from no other neurological comorbidities, preferably with the proviso that the subject may suffer from autism.

4. The method according to any one of clauses 1-3 wherein the agent is lacosamide or a functionally equivalent analogue thereof, preferably lacosamide.

5. A method for treating attention deficit disorder (ADD) or attention deficit hyperactivity disorder (ADHD) comprising administering a therapeutically active amount of lacosamide to a subject suffering from ADD or ADHD.

6. The method of clause 5 wherein the subject does not also suffer from epilepsy, bipolar disorder, any seizures, and/or neuropathic pain, particularly epilepsy and/or bipolar disorder

7. The method according to any one of clauses 1-6 wherein the therapeutically active amount is between 25-600 mg/day.

8. The method according to any one of clauses 1-7 wherein the therapeutically active amount is administered per the following treatment regimen:
(i) an initial amount of 50-100 mg/day, which if tolerated by the patient is followed by:
(ii) every 1-2 weeks increasing the amount by 50-100 mg up to a maximum amount in the range 200-600 mg/day and maintaining the daily amount thereat.

9. The method according to any one of clauses 1-8 wherein the therapeutically active amount is administered per the following treatment regimen:
(i) an initial amount of 50 mg/day, which if tolerated by the patient is followed by:
(ii) every 1-2 weeks increasing the amount by 50 mg up to a maximum amount in the range 200-300 mg/day and maintaining the daily amount thereat.

10. The method according to any one of clauses 1-9 wherein the therapeutically active amount is administered per the following treatment regimen:
(i) 50 mg/day for 1-2 weeks, followed by:
(ii) 100 mg/day for 1-2 weeks, followed by:
(iii) 150 mg/day for 1-2 weeks, followed by:
(iv) 200 mg/day for 1-2 weeks, followed by:
(v) 250 mg/day for 1-2 weeks;
(vi) maintaining the amount at 250 mg/day

11. The method according to any one of clauses 1-8 wherein the therapeutically active amount is administered per the following treatment regimen:
(i) an initial amount of 100 mg/day, which if tolerated by the patient is followed by:
(ii) every 1-2 weeks increasing the amount by 100 mg up to a maximum amount in the range 400-600 mg/day and maintaining the daily amount thereat.

12. The method according to any one of clauses 1-11 wherein the therapeutically active amount is between 1-12 mg/kg/day.

13. The method according to any one of clauses 1-12 wherein the therapeutically active amount is at least 1 mg/kg/day, at least 2 mg/kg/day, at least 4 mg/kg/day, at least 6 mg/kg/day, at least 8 mg/kg/day, or at least 10 mg/kg/day.

14. The method according to any one of clauses 1-14 wherein the therapeutically active amount is administered per the following treatment regimen:
(i) 2 mg/kg/day for 1-2 weeks, followed by:
(ii) 4 mg/kg/day for 1-2 weeks, followed by:
(iii) every 1-2 weeks increasing the amount by 2 mg/kg/day or less up to a maximum amount in the range 8-12 mg/kg/day and maintaining the daily amount thereat.

15. The method according to any one of clauses 1-14 wherein the therapeutically active amount is 250 mg/day or less.

16. The method according to any one of clauses 4-15 wherein lacosamide is administered in one dose per day.

17. The method according to any one of clauses 4-16 wherein lacosamide is administered in two or three doses per day.

18. The method according to any one of clauses 1-17, wherein the subject suffers from ADHD and/or the treatment is the treatment of ADHD.

19. The method according to clause 18 wherein the subject suffers from the combined type ADHD and/or the treatment is the treatment of the combined type ADHD.

20. The method according to any one of clauses 1-19 wherein the subject is a child, optionally wherein the child is 10 years old or less.

21. An agent for use in a method for treating attention deficit hyperactivity disorder (ADHD) or attention deficit disorder (ADD), the method comprising administering a therapeutically active amount of the agent to a subject suffering from ADHD or ADD, wherein the agent is active in the brain but does not stimulate neurotransmitter release or recapture transporters (in the brain).

22. The agent for use of clause 21 wherein the agent is lacosamide or a functionally equivalent analogue thereof.

23. The agent for use of clause 21 wherein the agent is lacosamide.

24. An agent for use of clause 21 wherein the subject does not also suffer from epilepsy, bipolar disorder, any seizures, and/or neuropathic pain.

25. The agent for use of any one of clauses 21 to 24 wherein the subject suffers from no other neurological comorbidities, preferably with the proviso that the subject may suffer from autism.

26. An agent for use in a method for treating attention deficit hyperactivity disorder (ADHD) or ADD, the method comprising administering a therapeutically active amount of the agent to a subject suffering from ADHD or ADD, wherein the agent is lacosamide.

27. The agent for use of clause 26 wherein the subject does not also suffer from epilepsy, bipolar disorder, any seizures, and/or neuropathic pain.

28. The agent for use according to any one of clauses 21-27 wherein the therapeutically active amount is between 25-600 mg/day.

29. The agent for use according to any one of clauses 21-28 wherein the therapeutically active amount is administered per the following treatment regimen:
(i) an initial amount of 50-100 mg/day, which if tolerated by the patient is followed by:
(ii) every 1-2 week increasing the amount by 50-100 mg up to a maximum amount in the range 200-600 mg/day and maintaining the daily amount thereat.

30. The agent for use according to any one of clauses 21-29 wherein the therapeutically active amount is administered per the following treatment regimen:
(i) an initial amount of 50 mg/day, which if tolerated by the patient is followed by:
(ii) every 1-2 weeks increasing the amount by 50 mg up to a maximum amount in the range 200-300 mg/day and maintaining the daily amount thereat.

31. The agent for use according to any one of clauses 21-30 wherein the therapeutically active amount is administered per the following treatment regimen:
(i) 50 mg/day for 1-2 weeks, followed by:
(ii) 100 mg/day for 1-2 weeks, followed by:
(iii) 150 mg/day for 1-2 weeks, followed by:
(iv) 200 mg/day for 1-2 weeks, followed by:
(v) 250 mg/day for 1-2 weeks;
(vi) maintaining the amount at 250 mg/day.

32. The agent for use according to any one of clauses 21-31 wherein the therapeutically active amount is administered per the following treatment regimen:
(i) an initial amount of 100 mg/day, which if tolerated by the patient is followed by:
(ii) every 1-2 weeks increasing the amount by 100 mg up to a maximum amount in the range 400-600 mg/day and maintaining the daily amount thereat.

33. The agent for use according to any one of clauses 21-32 wherein the therapeutically active amount is between 1-12 mg/kg/day.

34. The agent for use according to any one of clauses 21-33 wherein the therapeutically active amount is administered per the following treatment regimen:
(i) 2 mg/kg/day for 1-2 weeks, followed by:
(ii) 4 mg/kg/day for 1-2 weeks, followed by:
(iii) every 1-2 weeks increasing the amount by 2 mg/kg/day or less up to a maximum amount in the range 8-12 mg/kg/day and maintaining the daily amount thereat.

35. The agent for use according to any one of clauses 21-34 wherein the therapeutically active amount is 250 mg/day or less.

36. The agent for use according to any one of clauses 21-35 wherein the agent is administered in one dose per day.

37. The agent for use according to any one of clauses 21-35 wherein the agent is administered in two doses per day, or three doses per day.

38. The agent for use according to any one of clauses 21-37 wherein the subject suffers from no other neurological comorbidities, preferably with the proviso that the subject may suffer from autism.

39. The agent for use according to any one of clauses 21-38 wherein the subject is a child, optionally wherein the child is 10 years old or less.

40. The agent for use according to any one of clauses 21-38 or the method of any preceding claim wherein the therapeutically active amount of the agent is in the range of about 25-600, 50-400, 100-400 or 200-600 mg/day.

41. Use of an agent in the manufacture of a medicament for treating attention deficit disorder (ADD) or attention deficit hyperactivity disorder (ADHD), the method of treatment comprising, consisting essentially of or consisting of administering a therapeutically active amount of the agent to a subject suffering from ADD or ADHD, wherein the agent is active in the brain but does not stimulate neurotransmitter release or recapture transporters (in the brain), preferably wherein the agent is lacosamide or a functionally equivalent analogue thereof.

42. Use of lacosamide in the manufacture of a medicament for treating attention deficit disorder (ADD) or attention deficit hyperactivity disorder (ADHD), the method of treatment comprising, consisting essentially of or consisting of administering a therapeutically active amount of lacosamide to a subject suffering from ADD or ADHD.

43. Use according to clause 41 or 42, wherein the subject and/or dosage regime is as defined in any of the preceding clauses.

Unless defined otherwise, all technical and scientific terms used herein have the same meanings as commonly understood by one of ordinary skill in the art to which this invention belongs. All publications and patents specifically mentioned herein are incorporated by reference in their entirety for all purposes in connection with the invention.

As used herein, the terms “including” and “comprising” are open-ended terms and should be interpreted to mean “including, but not limited to.” On the other hand, the term “consisting of” should be understood to mean “including and limited to”.

As used herein and in the appended claims, the singular forms “a,” “an,” and “the” include plural reference unless the context clearly dictates otherwise. As well, the terms “a” (or “an”), “one or more” and “at least one” can be used interchangeably herein.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number, and thus will typically refer to a number or value that is 10% below or above the specifically recited number or value.

The term “subject” may be utilized herein interchangeably with the terms “patient” or “individual”.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims. Moreover, all aspects and embodiments of the invention described herein are considered to be broadly applicable and combinable with any and all other consistent embodiments, including those taken from other aspects of the invention (including in isolation) as appropriate.

### EXAMPLES

The invention will be further understood with reference to the following experimental examples.

### Example 1: Case Study 1

Overview

ADD and ADHD have been accepted as a very frequent and highly represented syndrome in childhood neuropsychological developmental troubles. The origin of this trouble has not been identified. Hypothesis about its etiology are deficit in dopamine production/firing and/or deficit in noradrenaline production/firing. As a consequence, stimulation by very active enhancers of neuronal activity (mainly methylphenidate but more recently others) is the conventional treatment for ADD/ADHD.

Conventional treatments such as methylphenidate and other forms of major and general brain stimulators mask the most overwhelming part of ADD/ADHD symptomatology and certainly do not modify the origin of the trouble. Moreover, the utilization of these powerful stimulants of the central nervous system usually, in mid and long term application, leads to a decline in their efficacy and simultaneous arousal of a number of neuropsychological symptoms (stereotypes, psychomotor retardation, intellectual impoverishment, anxiety and the like).

It was our aim to identify the semiology categorization that could be related to a precise etiology. We then sought to identify a therapeutic route that would act on this syndrome in the long term.

We present the case study of a 7 year old child suffering from ADHD (with no comorbidity such as epilepsy or bipolar disorder). The child was treated with a lacosamide mono-therapy for several weeks based on the conventional treatment regime for lacosamide in children (for the treatment of epilepsy and/or bipolar disorder). We observe that ADHD symptomatology regressed dramatically in the patient, with a significant improvement in child's cognitive functionality and behaviour.

These results confirm the possibility of treating ADHD symptomatology without neuronal stimulants, contradicting the etiologic hypothesis that sustains current pharmacological therapeutics (stimulants).

Evaluation (Pre-Treatment)

This case study describes the case of a 7-year old male (“the patient”) with ADHD of the combined type. The patient had not previously been treated with any psychotropic drugs.

Before initiating the selected drug treatment, the patient underwent a full medical examination. In addition, reports from his school teachers as well as parents were collected.

During examination, the patient presented significant dysphasia, which prevented him from following the general sense of other people's expression, disturbing even more the possibility of his own expression. His parents indicated that the patient could barely identify and recall his parents' or sister's name. He struggled to write his own name. Physically, the patient appeared strong and functional, although his behaviour was indicative of some kind of instability.

No abnormalities were found during electroencephalography (no asymmetry, no epileptiform activity).

The patient was unable or hardly able to concentrate at school; he could not follow instructions in class, nor could he work on his own. When questioned by a teacher, he either refused to answer or made no sense. The patient was able to copy words, however he did not appear capable of a creative thought (e.g. create stories). In class, the patient was highly agitated, disoriented and interruptive to his fellow students. He was capable of working under constant supervision form the teacher, but unable to sustain the work when left alone.

The severity of his cognitive symptoms resulted in an audiological study to verify whether or not the patient was deaf. The results of this study confirmed functional hearing.

In general, child's mood was quite positive, however occasional mood swings, including irritability and aggression towards his sister, hyperactive episodes, depressive behaviour and hyperemotionality were observed. In addition, the patient presented with poor sleep quality (hypersomnia) with episodes of agitated sleep activity, enuresis, and frequent lethargic afternoon napping.

Based on the examination, school reports and the interview with the parents, the patient was diagnosed with ADHD of the combined type.

The Selected Treatment

Lacosamide is currently being used in the United States and Europe to treat seizures in patients with epilepsy. The patient did not suffer from epilepsy, but the inventor chose to try using Lacosamide to treat ADHD in the patient.

Treatment Administration

Lacosamide was administered to the patient following the approved treatment regimen for lacosamide (Vimpat®) in epileptic children from 4 years of age. This is shown in the table below.

TABLE 1
Treatment regimen for lacosamide

| | |
|---|---|
| Starting dose | 2 mg/kg/day |
| Titration (incremental steps) | 2 mg/kg/day every week |
| Maximum recommended dose in patients <40 kg | Up to 12 mg/kg/day |
| Maximum recommended dose in patients ≥40 kg to <50 kg | Up to 10 mg/kg/day |

The treatment was delivered twice a day and went on for >8 weeks. No secondary reactions or effects were observed.

Observations

Days 1-10: Dose of 25 mg Every 12 Hours (50 mg/day)

Day 8—Observation

The patient tolerated the dose well—no allergic reactions were observed, with the exception of sporadic headaches, which ceased after the first few days of treatment.

No improvement to patient's behaviour, poor sleeping activity, or cognitive functionality at home or school was recorded. In consultation, the patient was unable to consistently read the syllable “ma”, nor was he able to communicate effectively.

Days 11-18: Dose of 50 MCI Every 12 Hours (100 mg/day)

Day 15—Observation

The patient presented an improved behaviour towards his sister in situations which would normally cause irritability or aggression. The patient was described as “calmer” by both the teachers at school and his parents. Parents indicated that the patient appeared more attentive to his school duties (e.g. doing homework). No improvement to the quality of sleep or cognitive functionality was recorded.

Days 19-28: Dose of 75 mg Every 12 Hours (150 mg/day)

Day 23—Observation

Improvement to the quality of sleep was recorded. The patient showed decreased enuresis and movement during sleep. In addition, cognitive functionality was improved. The patient was able to identify his father's name, pronounce the words more clearly and recognise nearly all letters in the alphabet. The patient also appeared less disorientated, having improved perception. At school, the patient was reported to recognise and name geometrical shapes. However, the patient was unable to understand and/or vocalize the meaning of a passage from a book that he read.

Days 29-38: Dose of 100 mg Every 12 Hours (200 mg/day)

Day 32—Observation

After the dosage was increased to 200 mg/day, lethargic napping in the afternoon was initially replaced with agitation and hyperactivity. These symptoms subsequently became milder. Up to this point, in any consultation carried out as part of this study, the patient was not open to conversation.

Day 36—Observation

The patient showed consistent improvement of his behaviour at school and home. He was calmer, less agitated and more socially aware. However, he did have one occurrence of an aggressive behaviour towards a fellow student. The patient was effectively attending to his school duties (e.g. doing homework). This was acknowledged in his school report.

During cognitive testing, the patient created a detailed story using elements of different stories that he has come across. The patient was able to sustain longer conversation, although often incoherent.

Days 39-63: Dose of 125 mg Every 12 Hours (250 mg/day)

Day 49—Observation

At school, the patient was consistently capable of working in a group, while remaining calm. At home, the patient did not show any aggression or irritability.

In consultation, the patient was more open to conversation, appeared to move with purpose. For the first time, the patient was able to sustain longer logical conversations, at least partially. The patient was more creative in the selection of words. After an explanation of a mathematical pattern, not only he could understand it, he could also reproduce it without any supervision from an adult. The patient tested positive for being able to consistently read the syllable “ma”.

Day 63—Observation

The patient showed a gradual improvement with regard to his dysphasia and was able to interact with others. The patient showed no signs of hyperactivity. No further behavioural incidents occurred. The quality of his sleep had dramatically improved—he slept alone and showed no difficulty with waking up. The cognitive functionality appears to gradually improve over time without any further changes to the dose of lacosamide.

Summary of the Results

Progression along weeks 1-2 was almost imperceptible.

The sleep pattern and vegetative functions began to stabilize after a dose of 150 mg/day (administered as two separate 75 mg doses).

Higher functions were stabilized closer to 200 mg/day (administered as two separate 100 mg doses): coherent phrases, pronunciation and precision in the intent of meaning—traits that typically would have been identified as language disorder. The levels of concentration in his school activities showed an increased comprehension capacity after treatment.

His social interaction improved, with the gradual disappearance of challenging or opposing attitudes as significant doses were administered. The quality of his participation in regular activities continued to improve through the development of the treatment protocol.

The doses were increased up to 250 mg/day without any persistent side effects and under regular controls. Regular visits were carried out throughout the treatment. To date, the maximum dose administered (250 mg/day) has shown no signs of intolerance or inadequacy.

Discussion

The improvement achieved in this case is not concerned exclusively with the control of an impulsiveness or hyperkinesia or with a suddenly recovered concentration, but with a set of improvements in the symptoms of the pathology.

Thus, lacosamide acts on a group of typical neuro-vegetative elements (lethargy, mood swings, involuntary movements of the limbs, among others); on behavioural components; and finally, on the various elements of the higher cognitive functions.

This is a progressive evolution. It must be highlighted that it opposes the one regularly described when stimulants are used in the treatment of this syndrome: these are characterized by an immediate and very significant action in its beginning, fading as the days pass. Here we observed quite the contrary: a slow and progressive improvement, increasingly more settled.

The follow-up of this case shows then, in the inventor's opinion, a surprising clinical finding, as it presents the following area of interest: considerable positive effects were achieved on the group of ADHD symptoms with the pharmacological intervention of a non-stimulant molecule.

This questions the well-known etiological hypothesis that indicates the need of a quasi-direct overstimulation of the dopamine/epinephrine pathway to try and re-establish what the brain of people suffering from ADHD would be unable to create on its own.

The issue of etiology may thus find its fundamental purpose: while different descriptions of the Deficit emphasize on its neurologic origin, none of them point to an etiology that was not determined by an imbalance in the production of one biogenic amine or another. Our results demonstrate the interest of an action almost purely ionic, which probably achieves its results in part due to its action on a new balance of those neuromediators but only as a result of an indirect action on these: obviously the action on the membrane will in turn correct the activity of different neuronal groups, and this will contribute to a balance closer to the desired one. By the way, this indirect action should enable us to sort out some issues on the type of sensibilisation produced by direct stimulants and that limit the efficiency of their action through time. That is to say, one of the problems with the utilization of stimulants not appreciated in the art is the sensibilisation that they will create when used—rendering the control of their action impossible over time. The methods of the present invention open the way to a mode of action which does not create sensibilisation.

### Example 2—Case Study 2

We conducted a second case study to validate earlier results of Case Study 1. The patient was a 17 year old male suffering from ADHD (with no comorbidity such as epilepsy or bipolar disorder). The patient was treated with a lacosamide mono-therapy for several weeks based on the conventional treatment regime for lacosamide (for the treatment of epilepsy and/or bipolar disorder) for subjects weighting 50 kg or more.

Similarly to Case Study 1, we observed that ADHD symptomatology slowly regressed in the patient, with a significant improvement in patient's cognitive functionality.

### Example 3—Case Study 3—Large Study Group

Importance

The aim of the study is to identify the viability and effectiveness of lacosamide in the treatment of symptoms of ADHD. Lacosamide is believed to work without stimulation and artificial modification of the concentration of neuro mediators. Instead, the compound works exclusively on the stabilisation of the ionic passage (Sodium, Potassium) through the neuronal membrane.

Several studies have demonstrated the effectiveness of methylphenidate and other neuro-stimulants in the treatment of ADHD. However, little is known about the effect of non-stimulating molecules, such as lacosamide, on the alleviation of symptoms associated with ADHD.

Objective

The present study is a series of cases in a group of children and adolescents with behavioural troubles, dysfunctional learning disabilities and ADHD syndrome, who have been treated using lacosamide and evaluated over the period of six months.

Methods

This study describes a series of cases of 12 children and adolescents (aged between 7 and 17 year old) treated with lacosamide. The patients were from 3 different orphanages and under the general supervision of a tutor. Importantly, the patients did not suffer from epilepsy or bipolar disorder or any psychiatric disorder.

We administrated doses adjusted to age and weight of each patient, in line with the therapeutic doses described herein. Specifically, lacosamide was administered to the patients following the approved treatment regimen for lacosamide (Vimpat®) in epileptic children from 4 years of age as per Table 1 above. The patients were at constant supervision of the tutor and other personnel of the orphanages.

The administration of lacosamide was checked weekly by the Study Monitor and GP.

Our study focused on the evaluation of the effects of lacosamide on the alleviation of ADHD symptoms.

Initial Selection of Patients

The selection of the children and adolescents was done after the positive response to 3 of the 4 initial examinations. Patient selection inclusion went through the following process:

1—Spontaneous Selection from the Tutor

Tutors were asked to choose children they already knew would test positive in the classic questionnaire (DSM5). The questionnaire tests for major learning difficulties, behaviour situations, lack of attention, impulsivity and hyperactivity (all characteristic of ADHD).

2—Clinical Interview+‘PASA’ Test

After being selected by the tutors, children were evaluated during a clinical consultation carried out in their own institution. Through this consultation, we verified the clinical condition of each child or adolescent, their records and anamnesis, if any. We also filled out the Clinical story forms. At the same time, we performed the first ‘PASA’ Evaluation. The ‘PASA’ Test is a clinical questionnaire that was developed by the inventor of the present application (see FIG. 1 for questionnaire questions). It includes examination of Thinking (“P” for “Pensamiento”), Mood (“A” for “Animo”), Sleep (“S” for “Sueno”) and Alterity (“A” for “Alteridad”).

3—Pedagogical Evaluation

In order to evaluate the academic level of the patients and later evolution while being treated, we established indicators of literary and mathematical skills of the patients in line with the official assessment of the Ministry of Education of the Dominican Republic.

4—Neuropsychological Tests

Each patient included in the study underwent neuropsychological evaluations, performed by an independent neuropsychologist, who did not perform the clinical interview, the ‘PASA’ test and the pedagogical evaluation. These tests offered an objective element that contributed to the establishment of the diagnosis.

Study

The selected patients underwent weekly consultation with a clinical psychologist (principal investigator) and a medical doctor (study monitor).

‘PASA’ Evaluation was repeated every 60 days after initiating the medication. The four editions of this questionnaire were compared and analysed, drawing a close-up portrait of the clinical evolution of these cases.

Academic evolution was assessed 90 days after initiating the treatment and at the end of the 6th month.

Neuropsychological evaluation was repeated after six months on medication, at the end of the study. These evaluations were guided by trained neuropsychologists. Four different evaluations were conducted: Continuous Performance Test (CPT)1, STROOP-P test, Wisconsin Card sorting Test and the Non Verbal Intelligence Test.

The pre- and post-treatment results were compared and analysed.

The Tutors Pre-Post Evaluation (self-administrated) was performed at the end of the study. This assessment required the person responsible for the patient's everyday life and care to evaluate the progression through the period of the study, including intellectual and behavioural changes.

To formalise the control of the treatment, a tool was designed, allowing to record all administrated doses to participants on a daily basis, including any occurrence or increase of adverse reactions or side effects. Every participant had an assigned and duly labelled weekly pillbox.

Presentation of the Problem

The available treatments for ADHD promote extracellular production of neurotransmitters. These stimulating compounds usually trigger the following patient's response:
an initial immediate or almost immediate improvement in the ADHD symptoms;
a second phase, where the initial and sudden improvement begins to deteriorate and secondary symptoms appear (insomnia, nervousness, tiredness, overexcitement, etc); and
a third phase of a more intense degradation (anguish, mood changes, bipolarity, hallucinations, etc.).

Without wishing to be bound by theory, the inventor believes that the above response profile can be explained by a double mechanism in neurones: the stimulation of a neuronal system produces a relieving and equilibrating effect corresponding to the unexpected and surprising effect—a general boost accompanied by the rearrangement of interneuronal transmitters charges. However, when the simulation is administrated regularly and repeatedly (as it would be as part of the ADHD treatment), tolerance and sensitisation are observed. Prolong sensitisation creates a neuroplastical adaptation to the treatment in the brain. These two effects of the stimulating compounds make them unsuitable for the ADHD treatment.

In view of the above, the inventors of the present application have tested the effect of a non-stimulant molecule (lacosamide) on the symptomatology of attention deficit disorder.

Results

Sociodemographic Aspects

In this study, 12 cases of children and adolescents were recruited within three orphanages.

‘PASA’

The overall trend is very favourable in all cases. Patients' symptoms improved in all categories. In this study, the patients were evaluated before treatment with lacosamide (evaluation 1) and every 2 months in the course of the treatment.

Clinical Evaluation of the Trend of Thought

The quality of the “Trend of Thought” is a very sensitive indication of an internal uncomfortable and disruptive trend of thoughts. It is a reliable item in a clinical evaluation of ADHD.

Overabundant (83%) and Disordered (67%) were the main forms of disruption identified by the included children; they both completely disappeared by the end of the study, diminishing progressively in time (FIG. 2).

In addition, a small improvement in the Tachypsychia and Hypercritical thinking was recorded over the course of this study.

Cognitive Thinking

At the start of the study, patients reported suffering from “blocking thoughts”, “sudden disconnections” with the sense of what was explained to them, and ˜83% declared not to understand most of the time what was said to them in class or in many other situations. They also identified they could not stop their thoughts keep evolving on another topic while trying to concentrate on one.

Blocking thoughts were present in 50% of the cases at the beginning of the study, which reduced gradually in the course of treatment with lacosamide to 25% (FIG. 3).

Initially, 50% of the patients identified sudden off-sense disconnections (spacing out). This symptom decreased to 33% at the end of the study (FIG. 3).

Extremely common in this kind of cases is the inability of sustaining attention or understanding in front of a long phrase or a sentence that is not directly indicative. This form of dysphasia was present in 83% of the patients, reducing to 42% at the end of the study (FIG. 3).

Second thoughts disturbing concentration diminished from an initial 83% of the population to 42% at the end of the study (FIG. 3).

Thoughts Content

The thoughts of patients were divided into three categories: anodyne, anxious and distressing. 17% of the patients at the beginning of the study indicated distressing thoughts. At the end of the study, distressing thoughts were completely eliminated (FIG. 4).

Mood and Neuro-Vegetative Function

At the beginning of the study, a high number of patients reported hyper emotion, cenesthesia, mood changes, hypomania, lethargy and tiredness (˜75%). These symptoms were significantly reduced in the course of the treatment with lacosamide (FIG. 5). Specifically, mood change fell to 0%, hypomania to 16.7%, lethargy to 8.3%, cenesthesia to 8.3%, tiredness to 8.3%, hyper emotion to 25%, and irritability fell from 33.3% to 16.7%. Importantly, depressive unjustified feelings reduced gradually from 25% to 0% after six months of treatment with lacosamide.

Re-Establishing the Sleeping Disorder

The data for sleeping characteristics was divided into two charts showing increasing and decreasing patterns (FIGS. 6A and 6B).

FIGS. 6A and 6B show that the patients significantly improved in most test categories. Difficulties with falling asleep improved from 75% to 0%. Cenesthesic presences lowered from 75% to 8.3% at the end of the trial period. Sleep interruption lowered from 83% to 8.3%. Realistic dreams were not significantly affected by the administration of lacosamide.

Patients experienced an improvement in the general state after waking up, sleeping without interruptions and ease of sleep.

It must be noted that the “sleeping trouble” was not treated by any other complementary molecule to lacosamide. Thus, the reestablishment of the sleeping cycle in all patients results uniquely from the action of this molecule.

Analysis of the ‘PASA’ Evaluation Ensemble

Overall, the administration of lacosamide has a positive impact on all tested ‘PASA’ categories. The three cited fields (Sleep Activity, Thoughts, Mood) improved for all patients. In addition, all patients responded to the treatment without substantial side effects.

The results for Mood and Sleep in most patients indicate complete or nearly complete alleviation of the symptoms.

Without wishing to be bound by theory, the sleeping activity improvement and modulation of the thoughts disorder and overabundant expression suggest that lacosamide stabilises a neurological function, which helps to re-establish healthy brain regulation.

Importantly, the pattern of gradual improvement in the course of the treatment differs from the response observed when a stimulating compound is used to treat ADHD. The results of this study clearly demonstrate that ADHD can be treated with lacosamide, a non-stimulating compound.

Neuropsychological Evaluation

We measured pedagogical values and characteristics of the executive function (Reasoning, Cognitive Flexibility, Attentional function, Intellectual Quotient, impulse inhibition, Perseveration resistance) throughout the course of the treatment with lacosamide.

Toni-2

The patients were assessed using the Test of Nonverbal Intelligence (TONI-2), which is a language-free measure of abstract problem-solving ability. The percentage of patients scoring in high average and average values increased at the end of the tested period from 16.7% and 50% to 33.3% and 58.3%, respectively (FIG. 7).

WCST

The patients were assessed using the Wisconsin Card Sorting Test (WCST), which is used to measure higher-level cognitive processes, such as attention, perseverance, abstract thinking, and set shifting. It is particularly used in clinical fields to measure perseverative behaviours that refer to an individual's insistence on wrong behaviour.

In WCST Flexibility test, 41.7% of patients undergoing lacosamide treatment scored in the lowest two categories in the first evaluation (FIG. 8: Mild and Sever Deterioration). In the second evaluation, only 16.6% of patients scored in the lowest two categories. Meanwhile, Average and High Average scores accumulated 50% of the sample (vs. 25% in the first one evaluation). “NA” in FIG. 8 stands for “No answer”.

In WCST Reasoning test, 50% of patients scored in the lowest, Severe Deterioration, category in the first evaluation (FIG. 9). This value was improved to 25% during second evaluation. On the second evaluation 58.3% of the sample concentrated in the 3 highest categories—an improvement from 41.7%.

STROOP-P

The patients were assessed using the STROOP-P test, which is used to assess the ability to inhibit cognitive interference that occurs when the processing of a specific stimulus feature impedes the simultaneous processing of a second stimulus attribute, well-known as the Stroop Effect.

Although the improvement after treatment with lacosamide is small, some tendency towards improvement appears for patients that initially tested with Severe Deterioration (25% to 8.3% in the second test, see FIG. 10). In addition, a higher number of patients recorded Average result in the second test (25% comparing to 8.3% in the first evaluation). “NA” in FIG. 10 stands for “No answer”.

Intellectual Quotient

IQ was tested prior to and after treatment. Results are shown in Table 2.

TABLE 2
The results of the IQ test.

| | | | Academic | |
|---|---|---|---|---|
| | Age | Gender | Degree | IQ |
| Pre-treatment | 7 | M | 5th Primary | CI-111 |
| Post-treatment | | | | CI-111 |
| Pre-treatment | 13 | F | 7th Primary | CI-87 |
| Post-treatment | | | | CI-91 |
| Pre-treatment | 12 | F | 5th Primary | CI-91 |
| Post-treatment | | | | CI-95 |
| Pre-treatment | 15 | F | 3rd Primary | CI-82 |
| Post-treatment | | | | CI-82 |
| Pre-treatment | 17 | F | Bachelor | CI-108 |
| Post-treatment | | | | CI-114 |
| Pre-treatment | 10 | M | 5th Primary | CI-95 |
| Post-treatment | | | | CI-104 |
| Pre-treatment | 16 | F | 2nd High School | CI-80 |
| Post-treatment | | | | CI-90 |
| Pre-treatment | 11 | F | 6th Primary | CI-93 |
| Post-treatment | | | | CI-95 |
| Pre-treatment | 7 | M | 2nd Primary | CI-94 |
| Post-treatment | | | | CI-106 |
| Pre-treatment | 14 | M | 2nd High School | CI-99 |
| Post-treatment | | | | CI-112 |
| Pre-treatment | 12 | F | 5th Primary | CI-81 |
| Post-treatment | | | | CI-91 |
| Pre-treatment | 8 | M | 3rd Primary | CI-117 |
| Post-treatment | | | | CI-117 |

Lacosamide resulted in a global IQ increase of 60 points for 12 patients tested. In none of the patients did treatment result in a lowering of the IQ value.

77% of the patients treated with this molecule raised their IQ values: 40% accumulated a progression average of around 11±2 points; 33% progressed on average by four points. The remaining 23% maintained previous IQ levels. These values indicate high clinical significance.

Lacosamide Neuropsychological Results Analysis

Lacosamide treatment was able to increase the levels of non-verbal reasoning to the point of doubling the number of subjects who reached the “high average” level. This means a significant increase in the ability to generate, perceive, analyse, synthesise, analyse, recover, manipulate and transform visual patterns and stimuli, a very important cognitive ability for learning, but also for self-regulation of behaviour. In addition, we observe lacosamide treatment improving cognitive flexibility in the sample subjects. This means that these children now have a better ability to adapt to changes in their environment, greater tolerance to them and to the changes that affect their surroundings and their lives, being more powerful to contemplate different perspectives, enabling them to find more than one solution to the same problem and better tolerate changes in planes, being able to put yourself in the other's place more easily.

Lacosamide treatment was positive but to a limited extent on the ability to inhibit cognitive interference that occurs when the processing of a specific stimulus characteristic prevents the simultaneous processing of a second stimulus attribute. This was measured in both cerebral hemispheres, finding similar results. Lacosamide treatment had no impact on the variables of distraction and impulsivity, mediated by the frontal lobes. In fact, even when we report improvement through the indicator Attentional Difficulty, others such Visual Attention, Perseveration Resistance and Impulse Inhibition did not show the expected improvement with the applied treatment.

Pedagogical Evaluation

Children and adolescents included in the study were evaluated pedagogically to establish the effect of lacosamide on the academic progress.

The patients did not receive the typical academic support they should have normally received at their age. Every single kind of scholarship or learning process was interrupted after the first month.

Therefore, the obtained results are directly related to the administration of lacosamide.

Skills

In order to measure academic evolution we used different parameters, which are: Spanish language, Reading, Writing, Comprehension of their own reading, Mathematics, Reasoning, argumentation capacities and Problem solving.

Independently, we established at each evaluation the attained level of alphabetisation and the acquisition of literary skills every patient had, as shown in Table 3.

TABLE 3
Levels of literary skills.

| | Literary Skills |
|---|---|
| Pre Syllabic Level | No comprehension of the alphabetical principle, |
| | therefore cannot find the identification between |
| | grapheme-phoneme |
| Syllabic Level | The child can detect at least one sound of the |
| | syllable, mostly vowels or continuous |
| | consonants |
| Syllabic Alphabetical Level | The child starts detecting and is able to |
| | represent some syllables in a complete form |
| Alphabetical Level | The child can detect all sounds and represents |
| | them correctly by his/her own writing |

Only 25% of the sample was “Alphabetised” (i.e. at the “alphabetical level”) by the beginning of the study. That proportion increased to 75% and maintained that level until the end of the study. The remaining 25% was spread in between a persistent lowest “Pre-Syllabic” and “Syllabic Alphabetical” level.

Pedagogic Evaluations

Overall, the results of pedagogic evaluation show gradual progression in all tested competencies throughout the 6-month treatment with lacosamide (FIG. 11; test 1: pre-treatment, test 2: after 3 months of treatment, test 3: after 6 months of treatment).

We categorised the sample following the protocol of the local Ministry of Education's Evaluation tool, which will qualify students following the consideration of “Non Acquired”, “Towards Acquisition” or “Acquired” levels.

83% of the sample was unable (Non Acquired) to Read, Write or understand what they were reading (Comprehensive Reading) in the first evaluation, prior to the treatment. Remaining 17% had already acquired all these literary skills. By the end of the study 75% completely acquired the reading and writing skills (+60%), and 83% were able to understand what they were reading. There were no remaining subjects (0%) at the “non-acquired” level: The remaining group of 25% were at the intermediate level, “Towards Acquisition”.

The initial Mathematics test showed an even distribution: 60% were unable to “Reason or argument” or “Solve problems” in the first evaluation, an accumulated of 40% were distributed in between acquisition and acquired skills. We found 50% of patients completely acquired (doubled initial 25%) “Reasoning” and “Solving Problems” after 3 months of treatment with lacosamide.

In conclusion, lacosamide not only positively affects the overall behaviour, mood and sleeping pattern of the patient, but also patient's intellectual and academic progress.

Tutors Evaluation Pre to Post Treatment

The tutors have evaluated the patients using the DSM-V questionnaire, which is typically used as a standard to classify mental disorders by mental health professionals in the United States. The results of the evaluation are shown in FIG. 12. Patients significantly improved across all tested elements. These results provide an independent assessment of the effect of lacosamide on the behaviour of the patients and confirm the results obtained in other tests conducted as part of this study.

Lacosamide is clearly a very powerful solution for patients suffering from ADHD.

1. An agent for use in a method for treating attention deficit hyperactivity disorder (ADHD) or attention deficit disorder (ADD), the method comprising administering a therapeutically active amount of the agent to a subject suffering from ADHD or ADD, wherein the agent is lacosamide or a functionally equivalent analogue thereof selected from lacosamide S-enantiomer, lacosamide racemate, or rufinamide.

2. The agent for use of claim 1 wherein the agent is lacosamide.

3. The agent for use of claim 1 or 2 wherein the subject suffers from ADHD, preferably of the combined type (ADHD-C).

4. The agent for use of any one of claims 1 to 3 wherein the subject does not also suffer from epilepsy, bipolar disorder, any seizures, and/or neuropathic pain.

5. The agent for use of any one of claims 1 to 4 wherein the subject suffers from no other neurological comorbidities, preferably with the proviso that the subject may suffer from autism.

6. The agent for use according to any one of claims 1-5 wherein the therapeutically active amount is between 25-600 mg/day.

7. The agent for use according to any one of claims 1-6 wherein the therapeutically active amount is administered in progressively increasing doses.

8. The agent for use according to any one of claims 1-7 wherein the therapeutically active amount is administered per the following treatment regimen:
(i) an initial amount of 50-100 mg/day, which if tolerated by the patient is followed by:
(ii) every 1-2 weeks increasing the amount by 50-100 mg up to a maximum amount in the range 200-600 mg/day and maintaining the daily amount thereat.

9. The agent for use according to claim 8, wherein part (ii) of the treatment regimen involves making the increase every week.

10. The agent for use according to claim 8, wherein part (ii) of the treatment regimen involves making the increase every 2 weeks.

11. The agent for use according to any one of claims 1-10 wherein the therapeutically active amount is administered per the following treatment regimen:
(i) an initial amount of 50 mg/day, which if tolerated by the patient is followed by:
(ii) every 1-2 weeks increasing the amount by 50 mg up to a maximum amount in the range 200-300 mg/day and maintaining the daily amount thereat.

12. The agent for use according to any one of claims 1-11 wherein the therapeutically active amount is administered per the following treatment regimen:
(i) 50 mg/day for 1-2 weeks, followed by:
(ii) 100 mg/day for 1-2 weeks, followed by:
(iii) 150 mg/day for 1-2 weeks, followed by:
(iv) 200 mg/day for 1-2 weeks, followed by:
(v) 250 mg/day and maintaining the daily amount thereat.

13. The agent for use according to any one of claims 1-11 wherein the therapeutically active amount is administered per the following treatment regimen:
(i) an initial amount of 100 mg/day, which if tolerated by the patient is followed by:
(ii) every 1-2 weeks increasing the amount by 100 mg up to a maximum amount in the range 400-600 mg/day and maintaining the daily amount thereat.

14. The agent for use according to any one of claims 1-13 wherein the therapeutically active amount is at least or about 1 mg/kg/day, at least or about 2 mg/kg/day, at least or about 4 mg/kg/day, at least or about 6 mg/kg/day, at least or about 8 mg/kg/day, or at least or about 10 mg/kg/day, preferably between 1-12 mg/kg/day.

15. The agent for use according to any one of claims 1-14 wherein the therapeutically active amount is administered per the following treatment regimen:
(i) 2 mg/kg/day for 1-2 weeks, followed by:
(ii) 4 mg/kg/day for 1-2 weeks, followed by:
(iii) every 1-2 weeks increasing the amount by 2 mg/kg/day or less up to a maximum amount in the range 8-12 mg/kg/day and maintaining the daily amount thereat.

16. The agent for use according to any one of claims 1-15 wherein the therapeutically active amount is 600 mg/day or less, such as 500, 400, 350, 300, or 250 mg/day or less.

17. The agent for use according to any one of claims 1-16 wherein the agent is administered in 1-3 doses per day, preferably one dose per day.

18. The agent for use according to any one of claims 1-17 wherein agent is administered in two doses per day, or three doses per day.

19. The agent for use according to any one of claims 1-18 wherein the subject is a child, optionally wherein the child is 10 years old or less.

20. The agent for use according to any one of claims 1-19, wherein the treatment results in one or more or all of the following:
(i) an improvement of cognitive function;
(ii) an improvement of cognitive performance;
(iii) a reduction in hyperactivity;
(iv) an improvement in the circadian rhythm and/or sleep quality; and/or
(v) an improvement in mood.

21. A method for treating attention deficit disorder (ADD) or attention deficit hyperactivity disorder (ADHD) comprising administering a therapeutically active amount of an agent to a subject suffering from ADD or ADHD, wherein the agent is lacosamide or a functionally equivalent analogue thereof.
